(19) United States
(12) Patent Application Publication
(10) Pub. No.: US 2023/0133374 A1
(43) Pub. Date: May 4, 2023

(54) METHOD, COMPUTER PROGRAM, SYSTEM AND VENTILATOR, FOR DETERMINING PATIENT-SPECIFIC RESPIRATORY PARAMETERS ON A VENTILATOR

(71) Applicant: Technische Universität München, München (DE)

(72) Inventors: Kei Wieland Müller, München (DE); Jonas Biehler, München (DE); Karl-Robert Wichmann, München (DE); Wolfgang Wall, Eching (DE)

(21) Appl. No.: 17/995,694

(22) PCT Filed: Apr. 8, 2021
(86) PCT No.: PCT/EP2021/059145

§ 371 Date: Oct. 6, 2022

(30) Foreign Application Priority Data
Apr. 9, 2020 (DE) 10 2020 204 632.8

(51) Int. Cl. A61M 16/00; A61B 5/087

(52) U.S. Cl. CPC A61M 16/026; A61B 5/087; A61M 2230/205

(57) ABSTRACT

The invention relates to a computer-implemented method, a computer program, a system, and a ventilation machine for determining patient-specific ventilation parameters for setting a ventilation machine by means of which the patient is to be ventilated.

The invention relates to a computer-implemented method, a computer program, a system and a ventilation machine, for determining a patient-specific ventilation parameter for setting a ventilation machine by means of which the patient is to be ventilated.

In the case of serious diseases of the lungs and the associated lack of supply of blood with oxygen absorbed by the lungs or the insufficient reduction of carbon dioxide released via the lungs, the patient must in many cases be kept alive with the aid of devices which ensure adequate gas exchange between the environment and the organism, more precisely the blood of the organism, in order to maintain the vital functions. The most frequently selected aid for maintaining a sufficient gas exchange is the ventilation machine, which conveys a gas mixture with a precisely variable oxygen content into and out of the lung of the patient via a pressure- or volume-controlled pump device, for example on the basis of a cyclically repeating pressure or volume profile.

In some diseases of the lungs, such as acute respiratory failure or acute respiratory distress syndrome (ARDS), ventilation in the form of the variable parameters on the ventilation machine must be adapted precisely and individually to the respective patient. This personalization of ventilation is often so complex that, in the course of the repeatedly necessary readjustment of the parameters, respiration-induced damage to the lungs can occur, which can lead to the death of the patient. This problem also arises in particular in the case of severe cases of coronavirus (COVID-19) diseases. The fine adjustment of the ventilation parameters is the key to reducing the enormously high mortality rate in ARDS by up to 40% even in specialized clinics. The assessment of ventilation with regard to its harmfulness to the ventilated lungs is currently only possible indirectly and in very broad terms. The medical gold standard works with statistically validated ventilation values and reference variables as well as treatment recommendations within the framework of so-called lung-protective ventilation, which are tested heuristically on the patient on the basis of empirical values, i.e. are based on data from the past.

The object of the present invention is to systematically determine the ventilation parameter of a ventilation machine for mechanical ventilation of a patient in order subsequently to achieve the most efficient and gentle ventilation possible.

This object is achieved by the method according to claim 1, the computer program product according to claim 22, the system according to claim 24 and the ventilation machine according to claim 25.
The method according to the invention is used for the automated determination of optimum ventilation parameters θ_{b, opt} for the mechanical ventilation of patients, and comprises the computer-implemented steps, that is to say those which can be implemented by means of at least one data processing device:
   Providing a patient-specific digital lung model, inputting initial input ventilation parameters θ_{b, init} as ventilation parameters θ_{b,i}=θ_{b, init}
   Performing the following steps (i) to (iii) iteratively until a completion criterion is met, which checks the reaching of optimal patient-specific ventilation parameters θ_{b,i}=θ_{b, opt} and/or provides for the reaching of a predetermined number of iterations;
      i) Evaluation of the mechanical ventilation simulated on the lung model as a function of the ventilation parameter θ_{b,i} by determining the value of at least one patient-specific target function F=F(θ_{b,i}) from the lung model, wherein
      the target function F describes a lung reaction to the simulated mechanical ventilation as a function of at least one output parameter of the lung model,
      ii) Evaluating the at least one determined value of the function F on the basis of at least one predetermined reference value, and
   selecting at least one next ventilation parameter θ_{b,next}, using a selection method dependent on at least one previously used ventilation parameter θ_{b,i};
   iii) Using the at least one next ventilation parameter θ_{b,next} as ventilation parameter θ_{b,i} to determine F=F(θ_{b,i}=θ_{b,next}) in step i);
Providing the patient-specific optimal ventilation parameter θ_{b,opt}.

The invention is based on the idea of systematically determining the settings of a ventilation machine with regard to their suitability for the individual patient using a model-based, personalized, computer-based prediction before they are applied to the ventilation machine, so that the patient is finally ventilated as ideally as possible, i.e. gently and efficiently. The ventilation settings of the ventilation machine are, in particular, parameterized and can be described as a vector-valued variable (ventilation parameter θ_{b}). They are used as model input variables (step (i)) and are successively (iteratively in steps (i) to (iii)) improved in that they are changed successively downstream of step (i), that is to say from iteration to iteration, with the aid of a selection method which, in particular, does not make use of the lung model and which, in particular, generates less computational effort than the lung model and which can therefore be carried out more quickly, on the basis of an evaluation (ii) of the simulated or physical lung reaction or result values (output values of the model of the lung).

The method according to the invention is not to be confused with the therapeutic method of (artificial) ventilation of the patient by a ventilation machine, in particular including the steps of connecting or disconnecting the patient from a ventilation machine,—this therapeutic method is not the subject of the invention.

An optimum ventilation parameter θ_{b, opt} is considered to be, in particular, a ventilation parameter which leads to a ventilation or lung reaction F which is physiologically acceptable to the patient and which is checked by means of the medically indicated, predetermined reference values. Even if a ventilation parameter suitable for this purpose has already been found, the method can find ventilation parameters which are still further improved, that is to say lead to a gentler or more efficient ventilation, if the iterations (i) to (iii) are continued, for example until a predetermined number of iterations is reached.

The method is preferably set up, that is to say in particular programmed, for at least one iteration to be carried out, that is to say for the method to return to step (i) at least once after step (iii). The method is preferably set up, i.e. in particular programmed, in order to return to step (ii) at least once after step (iii). The method is preferably set up, i.e. in particular programmed, in order to select at least one next ventilation parameter θ_{b,next} and to use it as an input variable for the lung model. The method is preferably set up, that is to say in particular programmed, for the optimization pass (i) to (iii) of the optimization loop (iteration) to be carried out at least once, twice, preferably K times, for example K=10, 15, 20, 25, 30, 50, 100.

The method according to the invention, in particular the lung model and/or the selection method, is preferably set up, i.e. in particular programmed, to determine one or more optimum ventilation parameters θ_{b, opt} in the shortest possible time. In this way, computing power is saved, or the technical outlay for implementing the method according to the invention is reduced.

The prediction of the optimum ventilation parameter according to the invention uses a personalized calculation model of the lung which serves to generate data relating to the suitability and selection of the best possible ventilation settings for the patient. A digital lung model suitable for carrying out the invention is described in the document by “C. J. Roth et al., A comprehensive computational human lung model incorporating inter-acinar dependencies: Application to spontaneous breathing and mechanical ventilation, Journal of Numerical Methods in Biomedical Engineering 2016; e02787 DOI: 10.1002/cnm.2787”. This lung model will be described below. However, the invention can also be carried out with modifications of this lung model, or with any other digital lung model which is suitable for quantifying the reaction of the lung to the ventilation, that is to say as a function of the ventilation parameters of a ventilation machine, as a target function F.

The lung reaction or target function F calculated from the lung model is patient-specific since the lung model is patient-specific. The lung model is patient-specific in particular because it was obtained on the basis of measurement data of the patient's lung, in particular was obtained on the basis of image data of the patient's lung, and/or was adapted by setting at least one lung model parameter θₘ to a patient-specific property, in particular to the physical property of the lung of a patient (see calibration), by means of a patient or disease history or genetic features. In particular, before the method is started, the lung model, which may have been determined, in particular, on the basis of image data of the patient's lung, is calibrated by means of a ventilation curve of the patient, which has at least one breath of the patient, and/or a special ventilation maneuver, such as, for example, a low-flow maneuver, and/or an esophageal pressure measurement. A ventilation curve in the sense of the invention consists of a pressure-time curve p_{trachea} (t) and/or a flow-time curve Q_{trachea}(t) and/or a volume-time curve v_{trachea}(t) and/or a respiratory gas mixture composition-time curve or curves derived therefrom or, in particular, combinations of these measurement curves. The curves result from the adjustment of the respirator and the metabolic, biological, biochemical, in particular physical response behavior of the patient, for example. During calibration, at least one, several or all parameters θₘ are adjusted. In particular, the material parameters of the alveolar clusters (ACs) and/or other material parameters. In particular, a patient-specific, volume-dependent pleural pressure boundary condition can also be calibrated, as well as further parameters θₘ.

For a person skilled in the art, reference is made, for example, to “C. J. Roth et al., Coupling of EIT with computational lung modeling for predicting patient-specific ventilator responses, Journal of Applied Physiology 122: 855-867, 2017; DOI: 10.1152/japplphysio1.00236.2016” from the information on page 856 ff. “Method Part” the model parameters θₘ, which are used for a calibration, can be taken.

Preferably, the at least one lung model is patient-specific, in that it has been selected in particular by a physician for this patient from a database of predetermined lung models which can be subdivided into categories, for example as a function of sex, age, weight, disease and/or body state, the physician performing the assignment of the patient to one of these categories of lung models, so that the lung model can (also) be used for the individual patient.

The lung model is referred to in particular as digital because at least one physical property of the lung is described by data and/or at least one algorithm.

The at least one target function F is preferably a target function B and/or a target function N, where N describes the enrichment of gas in the blood of the lung as a function of at least one output parameter of the lung model, where the at least one output parameter describes a gas partial pressure in the blood of the patient, and B describes the mechanical loading of the lung as a function of at least one output parameter of the lung model, where the at least one output parameter describes a mechanical loading variable of the lung of the patient. The method preferably uses exactly the two target functions B and N. However, it is also possible and preferred for the method to use other target functions or, instead of B or N, at least one other or further target function for describing a lung reaction.

Function B preferably describes the mechanical load caused by ventilation by means of an strain ε(x, t) of the lung tissue and/or a pressure p(x, t) prevailing in the lung. The function B preferably describes a dependence on the strain B(ε(x, t)) and/or the pressure B(p(x, t)) in the lung. Preferably, Function B additionally describes, or a further function B describes, the mechanical load caused by the ventilation by means of the collapse c(x, t) and/or the reopening r(x,t) (also referred to as “re-opening”) of the airways and/or alveoli and/or a surface-active factor of the alveoli sf(x, t). Preferably, function B additionally, or B₂ additionally, or a further function B₃ describes the mechanical load caused by ventilation by means of an surface-active factor of the alveoli sf(x, t), in particular of a lung surfactant. The lung surfactant is known in particular as a surface-active substance produced by pneumocytes type II in the lung and secreted on the surface of the alveolar epithelium as secretion.

The function N preferably describes at least the gas partial pressure of oxygen μ(O₂) and/or carbon dioxide μ(CO₂) in the venous or arterial blood of the lung caused by ventilation.

The selection method for selecting next ventilation parameter values θ_{b,next} preferably contains an algorithm, in particular an optimization method according to Bayes, which is implemented in particular using one or more Gaussian processes, random rests, artificial neural networks or other regression models, a fuzzy logic algorithm, an algorithm based on an evolutionary method, an algorithm containing a gradient method, and/or an algorithm based on stochastic techniques.

An optimization method according to Bayes is described by “Snoek, J. et al. Practical Bayesian Optimization of Machine Learning Algorithms, Advances in Neural Information Processing Systems, 2012”. Further descriptions, in particular of the regression models, can be found in “B. Shahriari, K. Swersky, Z. Wang, R. P. Adams, N. de Freitas, Taking the human out of the loop: A review of bayesian optimization, Proceedings of the IEEE, 2016; vol. 104, no. 1, pp. 148-175.” A Bayesian method applied to the present invention is described below.

Further algorithms, in particular an algorithm containing a gradient method, are described in Fletcher R., Practical Methods of Optimization, Print ISBN: 9780471915478, Online ISBN: 9781118723203, DOI: 10.1002/9781118723203, 1987 by John Wiley & Sons, Ltd. Algorithms based on stochastic techniques are described in Spall, J. C., Introduction to Stochastic Search and Optimization, 2003; Wiley. ISBN 978-0-471-33052-3.

The selection method for selecting at least one next ventilation parameter θ_{b,next} is preferably based on a probabilistic regression method, which depends on at least one previously determined data set Tᵢ=(θ_{b,i}, F(θ_{b,i})) and on an acquisition function for selecting next ventilation parameter values θ_{b,next}, in particular a regression method based on a Gaussian process.

The selection method for selecting at least one next ventilation parameter θ_{b,next}, preferably includes an acquisition function which uses the expected value of the improvement (“expected-improvement function”), in particular taking contrains into account (“expected-constrained-improvement function”).

The selection method for selecting at least one next ventilation parameter θ_{b,next}, preferably includes an acquisition function which uses an entropy search or which uses a knowledge gradient.

The ventilation parameter θ_{b,i} is preferably described by a set of parameters which describe the pressure-time profile and/or the volume-time profile of a breath, whereas θ_{b,i} is in particular selected from the group of possible and preferred parameters θ_{b,i}={pᵢₙₛₚ, PEEP, tᵢₙₛₚ, tₑₓₚ, tₚₗₐₜₑₐᵤ, f, FiO₂,\dot{p}ᵤₚ\dot{p}_{down}} profile. It is preferably provided in step (ii) to determine at least one next ventilation parameter θ_{b,next} preferably to determine a plurality of next ventilation parameters θ_{b,next} in particular by parallel calculation. The parameters of a ventilation parameter can have different values, also referred to in detail as “parameter values” and collectively also referred to as “ventilation parameter values”. In the context of this invention, “determination of a next ventilation parameter” means that the vector-valued, in particular unambiguously dimensioned, ventilation parameter consisting of a plurality of parameters is changed in such a way that its ventilation parameter values differ with respect to at least one parameter value from ventilation parameters already tested beforehand.

Preferably, in step (ii) of the method, the patient-specific function B is evaluated, taking into account the at least one constraint that the patient-specific function N does not fall below or exceed a predetermined reference value, wherein, in particular, N takes into account the output parameter “oxygen partial pressure μ(O₂)” and the constraint includes that the oxygen partial pressure μ(O₂) does not fall below the reference value S_{O2} of the enrichment of the oxygen enriched in the blood of the patient, and wherein, in particular, S_{O2}, is predetermined, in particular, as a patient-specific reference value. Evaluated within the meaning of the invention means, in particular, a comparison with previously obtained values for other ventilation parameters θ_{b,i} and/or a comparison with values or limits for B from the literature or medical practice.

Preferably, step (ii) of the method includes that, as a patient-specific reference value, in particular a maximum strain Bₘₐₓ(ε(x, t)) and/or pressure Bₘₐₓ(p(x, t)) within the lung must not be exceeded, and/or an oxygen saturation S_{O2} must not be undershot.

Preferably, in step (iii), at least one new ventilation parameter θ_{b,i} is determined by a Bayesian optimization step. Alternatively, the ventilation parameter can also be varied via systematic (stochastic or deterministic) methods. A systematic variation is preferable to a grid-search, since this results in a gain of efficiency or a shortened running time and reduced computational complexity of the method for finding suitable ventilation parameters.

Preferably, a set of initial input ventilation parameters θ_{b,1:J,init} is created by means of a random or quasi-random method, in particular Monte-Carlo or Latin hypercube sampling.

In step (ii), the function values of function B, which were calculated from the at least one output parameter by simulating the set of the initial input ventilation parameters θ_{b,1:J,init} are preferably used fro training a Gaussian model.

Preferably, in step (ii), the function values of function B calculated from the at least one output parameter by simulating the set of next input ventilation parameters θ_{b,next} are used for further training of the Gaussian model, in addition to the function values of function B obtained from the previous evaluations, so that the Gaussian model is successively trained on a larger amount of data. In other words, all determined function values contribute to determining the form of function B, so that the more and more trained Gaussian model increasingly accurately estimates which parameters are promising for success without having calculated function values in all areas of function B actually.

In step (ii), the selection function is preferably an acquisition function which selects at least one next ventilation parameter value θ_{b,next} taking into account an improvement function Ĩ(θ_{b,next}) and the improvement function is calculated as follows: Ĩ(θ_{b,next})=max {0, B(θ_{b}⁺)−\tilde{B}(θ_{b,next})}, wherein \tilde{B}(⋅) represents the posterior distribution of the surrogate model at the point θ_{b,next} and B(θ_{b}⁺) represents the function value with the lowest mechanical load so far as a function of the hitherto most suitable ventilation parameter θ_{b}⁺ and the acquisition function is composed as a product of the expected value of the improvement function Ĩ(θ_{b,next}), according to El= [symbol] [Ĩ(θ_{b,next})|θ_{b,next}] and an additional function \tilde{Δ}(θ_{b,next}) to form \tilde{Δ}(θ_{b,next})* [symbol] [Ĩ(θ_{b,next})|θ_{b,next}], where \tilde{Δ} results from an indicator function Δ which is 1 if the function N(θ_{b,next}) is smaller or larger than a predetermined reference value and zero otherwise, and the use of a probabilistic surrogate model Ñ(θ_{b,next}), in particular on the basis of a Gaussian process.

The patient-specific lung model is preferably patient-specific in that it has been produced as a function of measured image data of the lung of the patient. Preferably, the image data were obtained by an imaging method on the patient, in particular by means of computed tomography (CT), magnetic resonance imaging (MRI), ultrasound, X-ray or electro impedance tomography (EIT).

Preferably, the patient-specific lung model is patient-specific, in that it has been created by means of measurement data from patients, in that, preferably before the start of the method, the patient-specific lung model is calibrated by means of a ventilation curve of the patient, which has at least one breath of the patient, and/or a special ventilation maneuver, such as, for example, a low-flow maneuver and/or an esophageal pressure measurement. In this case, at least one, several or all parameters θₘ are calibrated, i.e. the values of the model parameters are determined at least partially by means of the ventilation parameters.

Preferably, the at least one predetermined reference value is patient-specific, in that it has been determined in particular by a physician for this patient, in that it is derived from medical empirical values for a patient category (sex, age, weight, disease, body state) to which the patient is attributed, or in that it has been determined by separate measurement on the body of the patient.

The invention also relates to a computer program product, using a digital lung model, comprising commands which, when executed on a processor of a data processing unit, have the effect that the following steps (i) to (iii) are carried out iteratively, until a final criterion is fulfilled which checks the reaching of optimum patient-specific ventilation parameters θ_{b,i}=θ_{b,opt} and/or checks the reaching of a predetermined number of iterations:
i) Evaluation of the mechanical ventilation simulated on the lung model as a function of the ventilation parameter θ_{b, i} by determining the value of at least one patient-specific target function F=F(θ_{b,i}) from the lung model, wherein
   the target function F describes a lung reaction to the simulated mechanical ventilation as a function of at least one output parameter of the lung model,
ii) Evaluating the at least one determined value of the function F on the basis of at least one predetermined reference value, and
   selecting at least one next ventilation parameter θ_{b,next}, using a selection method dependent on at least one previously used ventilation parameter θ_{b,i};
iii) Using the at least one next ventilation parameter θ_{b,next} as ventilation parameter θ_{b, i} to determine F=F(θ_{b,i}=θ_{b,next}) in step i).

The invention also relates to a computer-readable medium on which a computer program product is stored which uses a digital lung model and has instructions which, when they are executed on a processor of a data processing unit, have the effect that the following steps (i) to (iii) are carried out iteratively until a completion criterion is fulfilled which checks whether optimum patient-specific ventilation parameters θ_{b,i}=θ_{b, opt} have been reached and/or checks whether a predetermined number of iterations have been reached:
i) Evaluation of the mechanical ventilation simulated on the lung model as a function of the ventilation parameter θ_{b,i} by determining the value of at least one patient-specific target function F=F(θ_{b,i}) from the lung model, wherein
   the target function F describes a lung reaction to the simulated mechanical ventilation as a function of at least one output parameter of the lung
ii) Evaluating the at least one determined value of the function F on the basis of at least one predetermined reference value, and
   Selecting at least one next ventilation parameter θ_{b,next}, using a selection method dependent on at least one previously used ventilation parameter θ_{b,i};
iii) Using the at least one next ventilation parameter θ_{b,next} as ventilation parameter θ_{b, i} to determine F=F(θ_{b,i}=θ_{b,next}) in step i).

The invention also relates to a system comprising at least one data processing device and a computer program product, wherein the at least one data processing device is configured to execute the computer program product and, in particular, to exchange data for controlling the ventilation machine with a ventilation machine, wherein the computer program product uses a digital lung model, and the computer program product comprises commands which, when executed on a processor of the data processing device, have the effect that the following steps (i) to (iii) are carried out iteratively until a completion criterion is fulfilled which checks the reaching of optimum patient-specific respiratory parameters θ_{b,i}=θ_{b, opt} and/or checks the reaching of a predetermined number of iterations:
i) Evaluation of the mechanical ventilation simulated on the lung model as a function of the ventilation parameter θ_{b, i} by determining the value of at least one patient-specific target function F=F(θ_{b,i}) from the lung model, wherein
   the target function F describes a lung reaction to the simulated mechanical ventilation as a function of at least one output parameter of the lung model,
ii) Evaluating the at least one determined value of the function F on the basis of at least one predetermined reference value, and
   selecting at least one next ventilation parameter θ_{b,next}, using a selection method dependent on at least one previously used ventilation parameter θ_{b,i};
iii) Using the at least one next ventilation parameter θ_{b,next} as ventilation parameter θ_{b, i} to determine F=F(θ_{b,next}=θ_{b,next}) in step i).

The system preferably contains at least one ventilation machine, which is set up in particular for data exchange with the data processing device of the system. The data processing device of the system can be a component of the ventilation machine. The system preferably contains at least one measuring device for obtaining measurement data, in particular image data, in particular CT, MRI, X-ray, EIT or ultrasound data, from which the lung model can be determined, the measuring device being set up in particular for data exchange with the data processing device of the system. The data processing device of the system can be a component of the measuring device.

The invention also relates to a ventilation machine comprising at least one control unit and a data processing device which is suitable for reading in and executing at least one computer program product, and wherein the at least one data processing device is configured to supply data for controlling the ventilation machine to the control unit and/or to exchange said data with the control unit for controlling the ventilation of a patient, wherein the computer program product uses a digital lung model, and the computer program product comprises commands which, when executed on a processor of the data processing device, have the effect that the following steps (i) to (iii) are carried out iteratively until a completion criterion is fulfilled which checks the reaching of optimum patient-specific ventilation parameters θ_{b,i}=θ_{b, opt} and/or checks the reaching of a predetermined number of iterations:
i) Evaluation of the mechanical ventilation simulated on the lung model as a function of the ventilation parameter θ_{b, i} by determining the value of at least one patient-specific target function F=F(θ_{b,i}) from the lung model, wherein
   the target function F describes a lung reaction to the simulated mechanical ventilation as a function of at least one output parameter of the lung model,
ii) Evaluating the at least one determined value of the function F on the basis of at least one predetermined reference value, and
   Selecting at least one next ventilation parameter θ_{b,next}, using a selection method dependent on at least one previously used ventilation parameter θ_{b,i};
iii) Using the at least one next ventilation parameter θ_{b,next} as ventilation parameter θ_{b, i} to determine F=F(θ_{b,i}=θ_{b,next}) in step i).

Further preferred embodiments of the method according to the invention, of the computer program product according to the invention, of the system according to the invention and of the ventilation machine according to the invention can be gathered from the following description of the exemplary embodiments in conjunction with the figures and their description. Features of these subject matters of the invention can be derived in each case from the description of the other subject matters of the invention and their configuration. Identical components of the embodiments are identified essentially by the same reference numerals, unless otherwise described or stated in the context. The drawings show:

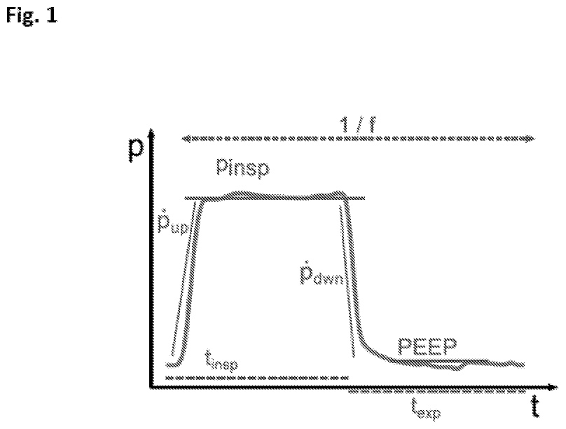

FIG. 1 schematically shows a pressure-time curve with a ventilation parameter selected by way of example, including parameters such as the end-expiratory pressure PEEP, the inspiratory pressure pᵢₙₛₚ, the pressure ramps \dot{p}ᵤₚ, \dot{p}_{down}, as well as the inspiratory and expiratory times tᵢₙₛₚ, tₑₓₚ.

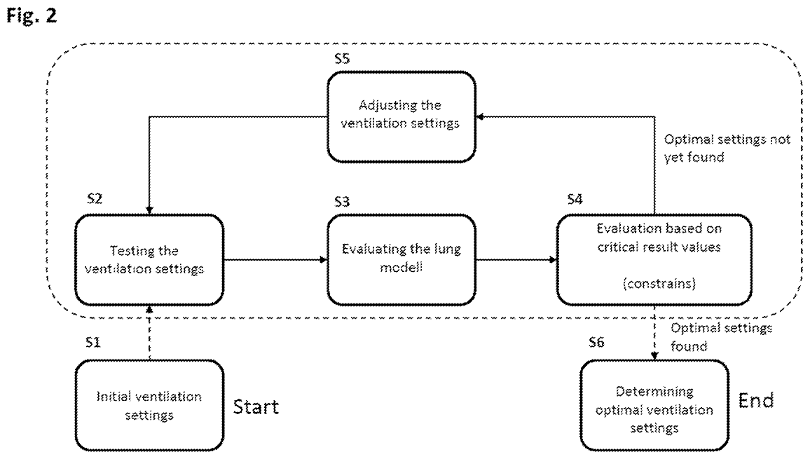

FIG. 2 schematically shows the principle of the iterative procedure for improving the ventilation parameter.

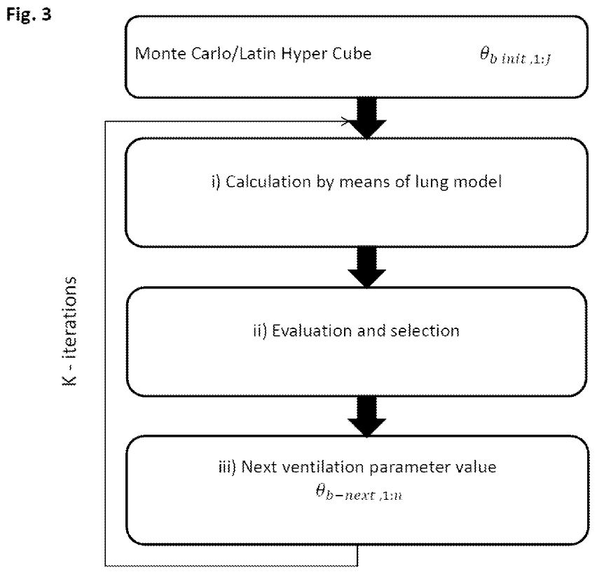

FIG. 3 shows steps i) to iii) for determining an optimum ventilation parameter, starting from providing of a set of initial ventilation parameters by means of Monte Carlo or Latin Hyper-Cube methods.

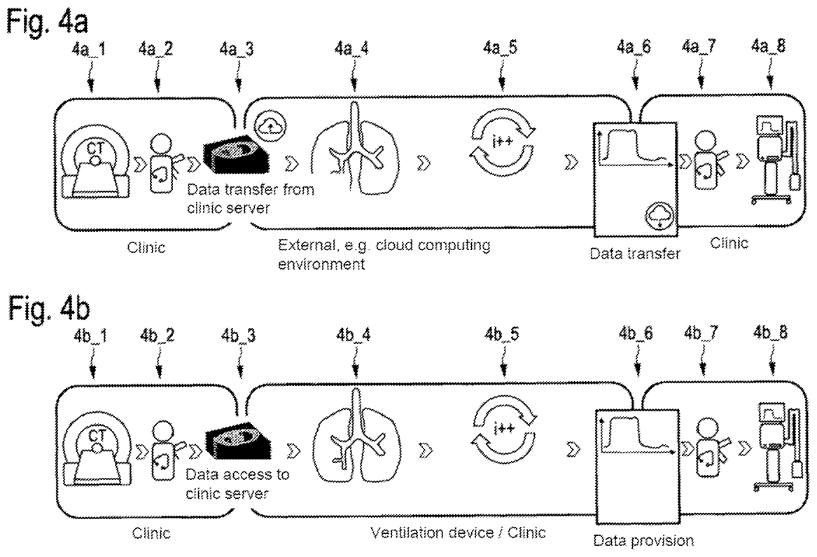

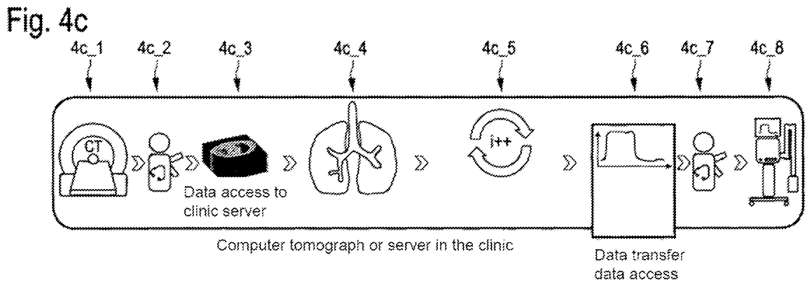

FIGS. 4a-c schematically show three different embodiments of the invention, in which, depending on the embodiment, the determination of the ventilation settings as a data logistic process chain takes place at different locations, in particular on a computing server of a cloud computing provider on a server in a clinic or in the ventilation device itself.

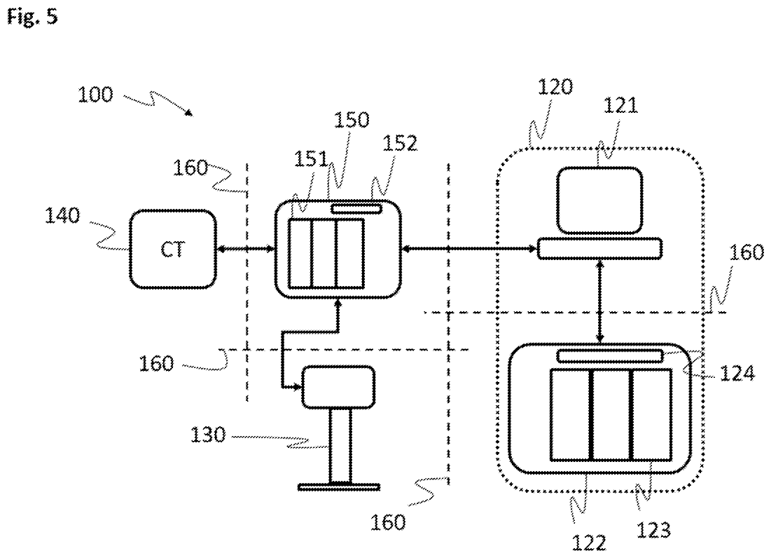

FIG. 5 schematically shows an embodiment of the system for determining an optimum ventilation parameter.

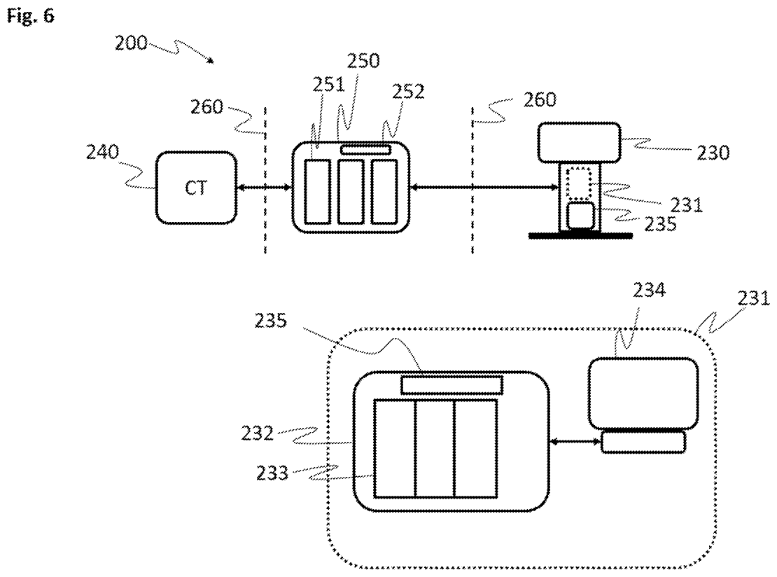

FIG. 6 schematically shows an embodiment of the ventilation machine, wherein the ventilation machine comprises a data processing device for carrying out the simulation and the optimization.

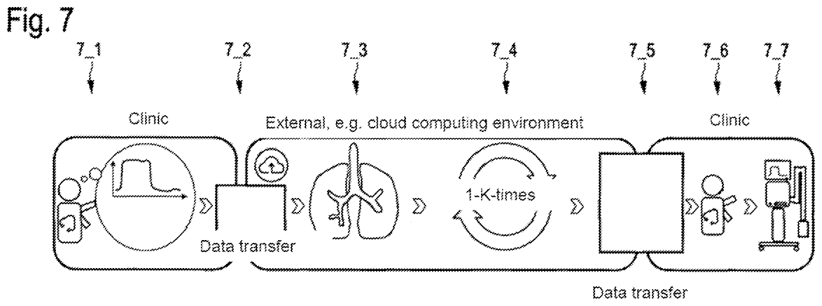

FIG. 7 shows an embodiment in which, without patient-specific imaging, a calculation model of the lung is parameterized purely on the basis of additional patient-specific data different from the imaging and is subsequently evaluated at least once in order to evaluate and improve the ventilation proposal of its user with regard to its suitability for the present patient. In this case, the 3D structural data would have to be adapted to the patient on the basis of the available data on the basis of an already existing “template”. For example, by taking into account factors such as body height and/or BMI. The 3D structural data could also be available in a kind of database and the data set which suits best to the patient would be selected.

### DETAILED DESCRIPTION OF THE INVENTION IN THE EMBODIMENT

A lung model is understood below as a digital, i.e. computer-implemented, model of a human lung which is suitable for simulating the physiology of a human lung. This may be a lung model which is based on the CT data of a patient, i.e. is specific to the patient. Alternatively, the lung model can be based on the evaluation of CT data of a patient group or generally on the evaluation of lung data from a database. A patient-specific lung model is also understood below as meaning a lung model which is calibrated to a patient, for example by calibrating the lung model by means of a real ventilation curve of the patient. The ventilation curve is in particular a pressure-time curve and/or flow-time curve of one or more breaths or ventilation maneuvers of the patient during artificial ventilation. The ventilation curve can be understood as a function of certain parameters characterizing the ventilation, i.e. the measured ventilation curve on the patient is displayed parameterized over a set of parameters. In particular, the most important parameters of the ventilation curve are the following:

PEEP, describes the pressure level at the end of exhalation (positive-end-expiratory pressure), Pᵢₙₛₚ, defined as inspiratory pressure describes the pressure level which specifies the target pressure during inhalation.

The two pressure ramps \dot{p}ᵤₚ, \dot{p}_{down}, describe the increase or decrease of the pressure during inhalation and exhalation, i.e. how quickly the pressure is increased or decreased.

The parameters tᵢₙₛₚ, tₑₓₚ describe the inhalation time and the exhalation time, i.e. how long is inhaled and exhaled.

The respiratory rate f or the period duration of a breath 1/f indicates how many breaths are taken during a unit of time, usually within one minute. The parameter FiO₂ describes the oxygen content in the respiratory gas. This value indicates how many gas percentiles in the respiratory gas mixture are oxygen. The specified parameters are not shown in full. Rather, the most important parameters which are set on a ventilating machine during pressure-controlled ventilation are mentioned here. However, volume-controlled ventilation can be used, for example. In this or other ventilation modes, the parameters are different. It is an object of the invention to improve these parameters, which can be set on a mechanical ventilator, in such a way, i.e. to optimize them in such a way, that the patient is ventilated by the ventilator in the most protective manner. Furthermore, parameters which cannot be set on the ventilator, can also influence the ventilation, such as, for example, the position of the patient (e.g. supine or prone position).

The input into the lung model comprises at least one ventilation parameter θ_{b,}, which contains the parameters of the ventilation curve θ_{b}={pᵢₙₛₚ,PEEP,tᵢₙₛₚ, tₑₓₚ,f,FiO₂,\dot{p}ᵤₚ,\dot{p}_{down}} in vector form. A ventilation parameter θ_{b} accordingly describes a multiplicity of respectively possible settings of the ventilation machine. In other words, the parameters {pᵢₙₛₚ,PEEP,tᵢₙₛₚ, tₑₓₚ,f,FiO₂,\dot{p}ᵤₚ,\dot{p}_{down}, . . . } span an input space of the mathematical model, which is mapped onto an output space of the model by simulation. It is an object of the invention to find in the input space those or the vector θ_{b,opt} or parameters by means of which output variables of the simulation model which are relevant to patients are minimized and/or maximized, taking into account predetermined and/or patient-specific specifications, i.e. reference values. These are, for example, the oxygen content and the carbon dioxide content in the venous and/or arterial blood of the patient.

Output variables of the simulation model of the lung can be, for example, the strain of the lung tissue ε(\vec{x},t), the pressure p(\vec{x},t)), the flow rates Q(\vec{x},t), and/or an surface-active factor of the alveoli sf(\vec{x},t) and/or a collapse c(\vec{x},t) and/or a re-opening r(\vec{x},t). The next input variable to be simulated, i.e. the next ventilation parameter θ_{b,next}, is determined as a function of the course of the simulated output variables relevant to the patient. In this case, a next ventilation parameter θ_{b,next} is selected in such a way that an ideal ventilation parameter θ_{b,opt} is found as quickly as possible, that is to say after a small number of iterations, that is to say simulation runs through the next ventilation parameter. Alternatively, in another embodiment, the model can also be aborted after a predetermined number of iteration steps, for example, and the ventilation parameter found to be most suitable up to that point can be output.

The lung model takes into account (i) the airways, which consist of the trachea, as well as the bronchi and bronchioles, (ii) alveolar clusters (AC), which comprise the alveoli and the alveoli connected to the alveolar sacs, as well as the proportions of the bronchioles contained therein, and (iii) the AC interaction, which takes into account the viscoelastic coupling of ACs adjacent to one another. When inhaled, the lung volume increases, which causes the alveoli to stretch, among other things. In this case, the alveoli which are adjacent to one another are linked in their expansion owing to the lung tissue which connects them to one another.

The model takes into account the three-dimensional geometric structure of a patient's lung. For this purpose, a data set which forms the 3D structure geometry of a patient lung is read into the lung model as an input variable. In one embodiment, the data set can alternatively be averaged over the structural geometry of a plurality of patients. For example, an averaged structural data set can be generated for patients with a certain pre-existing lung disease and can be read into the lung model in order to provide specific ventilation parameters for patients with this pre-existing lung disease. Likewise, the input ventilation parameters can represent parameter values averaged from a database. Based on the structural data set, the model constructs a digital image of the patient's lung. In one embodiment, the structure-geometric data set is based on 3D patient-specific CT data (computed tomography images, layer thickness and pixel size 0.7344 mm) of a 42-year-old male patient with a functional residual capacity (FRC) of FRC=2.65 |and a total lung capacity (TLC) of TLC=4.76 |. Since the model simulates the patient lung in its three-dimensional form, the mechanical properties, such as tissue strain and pressure distribution, can also be output as locally resolved output variables by the model.

The model is suitable for simulating the effect of local overstretching of the lung tissue, which can be caused, for example, by artificial ventilation, before this effect can be ascertained by means of measuring devices present in the clinic, or the patient is irreversibly damaged by suboptimal ventilation settings.

### Model-Based Representation of the Respiratory Tract

The airways are divided into the trachea and the bronchial system, which is divided into a right and a left main bronchial branch (main bronchus) and which supplies oxygen to one of the two lungs. Each bronchial trunk is further divided into smaller bronchi (bronchi of the second order): The right main bronchus usually branches into three main branches, which supply the usually three lobes of the right lung. The left main bronchus is usually divided into two main branches for the usually two lobes of the left lung. These five main branches form the so-called lobar bronchi, which branch out further to the segment bronchi and into ever smaller branches (generations). After about 20-25 division steps, i.e. generations, the widely branched system of the bronchial tree thus arises. This system of bronchi is available via the CT image data set and can be converted into a 3D structure data set or segmented, for example, by means of an image recognition algorithm based on artificial intelligence. This is then made available to the lung model for constructing the model geometry of the lung.

The smaller the bronchi become, the simpler and thinner their internal structure becomes. The smallest branches of the bronchi, the bronchioles, have an internal diameter of less than 1 mm. Therefore, the CT resolution is not sufficient to represent these structures in a spatially resolved manner. While the lower-generation airways are segmented directly from the CT data, the higher-generation airways are generated using a space-filling algorithm, as described, for example, in “Ismail M, Comerford A, Wall W A. Coupled and reduced dimensional modeling of respiratory mechanics during spontaneous breathing. International Journal of Numerical Methods in Biomedical Engineering 2013; 29:1285-1305”. The airways (trachea and bronchial tree) are generated recursively from the generation in which segmentation from the CT data is no longer possible or earlier, until the peripheral airways reach a length termination criterion (Iₜ=1.2 mm), a radius termination criterion (rₜ=0.2 mm) or a generation termination criteria (N_{gen}=17). The scaling of the radius of the daughter-to-parent branch of the left and right branch of the bronchial tree is 0.876 and 0.686, respectively, as is generally known from morphological studies of the human body. The radius scaling, the airway orientation and the airway length can be adapted as a function of the CT data spatially assigned to them in order to map the inhomogeneity of the lung. The segmented lower-generation respiratory tracts, which are based on the CT data, are connected to the higher-generation respiratory tracts, which are generated using the space-filling algorithm. For example, a digital lung model has a total of 60.143 airways, of which 30.072 are peripheral airways, i.e. airways of higher generations. The bronchioles (highest generations) branch once again into microscopically finest branches (Bronchioli respiratorii), which terminate in the acini. These acini eventually lead into the actual lung tissue responsible for the gas exchange with a total of about 300 million alveoli. In the lung model, one or more acini and the bronchioles contained therein are combined to form an AC. In the embodiment described here, the mathematical modeling of the airways is carried out by implementing a dimensionally reduced zero-dimensional (0-D) flow model, which is described in “Pedley T J, Schroter R C, Sudlow M F. The prediction of pressure drop and variation of resistance within the human bronchial airways. Respiration Physiology 1970; 9:387-405”. In this way, the mean flow behavior of the airways can be modeled efficiently and, for example, using reduced computing power. The model follows an approach in which the pressure difference ΔP along an airway is represented as a linearly dependent variable of the flow resistance R and of the flow rate Q through the respiratory tract channel as ΔP=Q*R. This expression is discretized according to ΔPⁿ⁺¹=Rⁿ⁺¹*Qⁿ⁺¹ where n=t/Δt. The by “Pedley T J, Schroter R C, Sudlow M F. The prediction of pressure drop and variation of resistance within the human bronchial airways, Respiration Physiology 1970; 9:387-405” formulated nonlinear flow resistance R takes into account both geometric and turbulent flow losses in the airway system of the lungs and was adapted to experimental lung data. In “Roth C. J. et al., A comprehensive computational human lung model incorporating inter-acinar dependencies: Application to spontaneous breathing and mechanical ventilation, Journal of Numerical Methods in Biomedical Engineering 2016; e02787 DOI: 10.1002/cnm.2787”, an extension is specified which takes into account the effect of the cross-sectional change of the airways during inhalation or exhalation.

### Model-Based Representation of the ACs

The ACs are generated by means of the inverse approach developed by “Ismail M, Comerford A, Wall W A. Coupled and reduced dimensional modeling of respiratory mechanics during spontaneous breathing. International Journal of Numerical Methods in Biomedical Engineering 2013; 29:1285 1305”. Since the CT data are in the end-expiratory state, a constant pleural pressure of 5.3 cm H₂O is assumed for the 3D lung geometry. According to the morphological data of the human alveoli size, which were measured at a transpulmonary pressure of 25 cm H₂O in generally known literature, and the inverse approach developed by “Ismail M. et al.”, a number Nₐₗᵥ of 797 million alveoli is determined in the lung calculated by way of example in this embodiment. The number of alveoli per lung lobe is

$${N}_{i}^{lb} = {N}^{alv} \frac{{V}_{i}^{lb}}{{V}^{lung}} ,$$

where Vᵢ^{lb} is the volume of a lobe and V^{lung} is the total volume of the lung. The number of alveoli on each peripheral airway is

$${N}_{i}^{alv} = {N}_{lb} \frac{{A}_{i}^{lb}}{{A}^{lb}} ,$$

where Aᵢ^{lb} is the exit area of the peripheral airway and A^{lb} is the sum of all exit areas of the peripheral airways within a lobe. For further specification, the number of alveoli Nᵢ^{alv} can be multiplied by a factor based on the CT data spatially assigned to the alveoli. Finally, the alveoli are grouped at each peripheral airway (so-called alveolar duct) to form an acinus. That is to say, an acinus is formed from a number of alveoli and alveoli ducts, the alveoli being grouped around the end of a respective alveoli ducts which supplies them with air for gas exchange. Physiologically, an alveolar duct thus always delivers air to a certain number of alveoli. Several alveolar passages form an acinus. One acinus or more acini, as well as bronchioles linking them, are combined to form an alveolar cluster. By means of this physiological grouping, the resolution of the model can be varied and makes it possible to implement the mathematical simulation in a simplified manner in the described embodiment.

In the embodiment described here, the mathematical conversion of the alveolar clusters is carried out by means of a method described in “Ismail M, Comerford A, Wall W A. Coupled and reduced dimensional modeling of respiratory mechanics during spontaneous breathing. International Journal of Numerical Methods in Biomedical Engineering 2013; 29:1285-1305”, which is based on a rheological model with Maxwell elements connected in parallel. This rheological model has been calibrated to implement the mechanical behavior of the rheology described in “Denny E, Schroter R C. Viscoelastic behavior of a lung alveolar duct model. Journal of Biomechanical Engineering 2000; 122:143-151”. For the sake of simplicity, it is assumed here that each AC is supplied with air from a respective airway and that all the alveolar passages contained therein behave identically. This approach also makes it possible to model the entire AC as a zero-dimensional (0-D) element, while retaining its viscoelastic properties. The resulting linear AC model is sufficient to correctly model the mechanical behavior of healthy lung tissue during spontaneous breathing. In order to be able to simulate both large lung tidal volumes and pressure variations with the lung model, the linear model has been extended to a non-linear model. For this purpose, the spring constant (linear spring model) describing the elasticity of the lung tissue of adjacent alveoli was replaced by a nonlinear expression which contains two exponential terms. This double exponentially stiffening law of materials is defined as follows:

E₁=E₁^{u}+E₁^{l};

E₁^{u}=E₁ᵢ⁰+b(vᵢ−vᵢ⁰)+k^{u}e^{τ}^{^{u}}^{(v}^{ᵢ}^{-v}^{ᵢ}^{⁰})

E₁^{l}=k^{l}e^{τ}^{^{l}}^{(v}^{ᵢ}^{-v}^{ᵢ}^{⁰}⁾

In this case, E₁ is the stiffness (spring constant), vᵢ is the volume of an alveolar duct and vᵢ⁰ is the volume of an alveolar duct in the stress-free state. Calibration of the T on simulated quasi-static p-V curves of the salt-washed alveolar duct as described in “Denny E, Schroter R C, Viscoelastic behavior of a lung alveolar duct model. Journal of Biomechanical Engineering 2000; 122:143-151”, and to a dynamic load at 0 Hz results in the following parameters:

E₁⁰=6.51×10⁻⁴ cmH₂O·cm⁻³

b=35.23×10⁷ cmH₂O·cm⁻⁶

k^{u}=6.79×10⁻⁵cmH₂O·cm⁻³

τ^{u}=14.47 cm·s⁻¹

k^{l}=5.32×10⁵ cmH₂O·cm⁻³

τ⁻¹=−9.0 cm·s⁻¹

In the embodiment described here, using further terms from “Ismail M., Comerford A, Wall W A. Coupled and reduced dimensional modeling of respiratory mechanics during spontaneous breathing. International Journal of Numerical Methods in Biomedical Engineering 2013; 29:1285-1305”, for example, the stiffness of the ACs as a function of the alveolar volume can be calculated and thus the pressure difference between the airway inlet of the AC and the environment can be determined as a function of the AC volume.

A classic Newton-Raphson scheme can be used for the solution of the completely coupled system of equations comprising nonlinear airways and nonlinear ACs, or a fixed-point iteration method or another suitable solution method is used.

### Model-Based Representation of AC Interaction

By means of alveolar cluster linker elements (ACL), the interaction of adjacent ACs and between ACs and respiratory tracts is modeled. These ACL link in pairs or in groups those ACs (and respiratory tracts) which influence each other. This interaction is caused, on the one hand, by the volume competition of the ACs within the lungs and, on the other hand, by the tissue that divides adjacent ACs. The resulting mutual influence is realized by additional forces at the ACs (and respiratory tracts) connected to the ACL. As a result, AC can be stretched even if the pressure is exerted only on the subpleural ACs. In other words, the boundary condition of the pleural pressure is only applied to the ACs actually adjoining the pleural gap.

This feature of the lung model plays an important role in heterogeneous lungs. Consequently, a mathematical description of the dependencies existing between the ACs in the lung model is a necessity for the realistic simulation of a patient-specific lung. A mathematical description of the implementation of these dependencies is given in “Roth C. J. et al., A comprehensive computational human lung model incorporating interacinar dependencies: Application to spontaneous breathing and mechanical ventilation, Journal of Numerical Methods in Biomedical Engineering 2016; E02787 DOI: 10.1002/cnm .2787”.

In the embodiment described here, the mathematical conversion of the ACL takes place as follows. ACLs are generated by determining all ACs and airways adjacent to an AC. For this purpose, an algorithm is used which, starting from one AC in each case, geometrically detects all ACs adjacent thereto, which can be achieved, inter alia, by means of a distance criterion or by means of neighborhood space-filling cells. Furthermore, those ACs are detected which are in direct connection with the lung-side pleura or the pleural space that is to say at least partially have no adjacent ACs. Once the neighborhood ratios of the ACs have been calculated, a coupling element is in-serted between the ACs (and the airways) as an “AC linker element”. This ACL coupler models the correct interaction between neighboring ACs by introducing it as a fictitious “inter-AC pressure” Pᵢₙₜᵣ, which ensures that pressure heterogeneities propagate across adjacent ACs. This allows the pleural pressure to be applied only to the subpleural ACs. This achieves a physiologically correct pressure distribution in the lung. Compared to earlier models, in which the pleural pressure is applied equally to all ACs, there is no deviation when the pleural pressure and the material properties, such as, for example, in a healthy lung, are homogeneously distributed. In the case of heterogeneous pleural pressure, for example due to the influence of gravity, and in the case of heterogeneous distribution of the material properties of the lung, forces which are exerted on an alveolar wall are distributed to the latter as a function of the number of adjacent ACs. Consequently, by introducing a fictitious “inter-AC pressure”, a patient-specific heterogeneous pressure and material property distribution can be physiologically simulated. In one embodiment, for example, 5981 ACs were determined which adjoin the pleural gap and a number of 140.135 ACLs were introduced accordingly.

### Calibration

The central aspects of the calibration of the model are described below. In the embodiment described here, the geometric structure of the patient-specific lung model is produced in a first step by means of image evaluation algorithms of a present set of computed tomographic slice images of a patient. The model can be considered abstractly as a function as follows, whereby in the present embodiment the output variables of the model are the pressure p(\vec{x}, t), the flow rate Q(\vec{x}, t), the strain ε(\vec{x}, t) of the lung tissue and the gas partial pressures p(\vec{x}, t) and Q(\vec{x}, t) are considerd:

[p(\vec{x}, t), Q(\vec{x}, t), ε(\vec{x}, t), μ_{CO}_{₂}(\vec{x}, t)]=M(θ_{b}, θₘ).

For given ventilation parameters eb and model parameters θₘ, the model thus provides a statement about the expansion, the pressure and various gas partial pressures as a function of the location x and the time t. The vectorial notation of the location vector will be dispensed with in the following. From these model output variables (which as a rule cannot be measured experimentally directly), other experimentally measurable variables, such as, for example, the tidal volume Vₜ of respiration (i.e. the actual volume breathed), can be calculated.

In the next step, the model parameters θₘ are adapted in such a way that the experimentally measurable tidal volume pressure curves V_{t;exp(t)} correspond to the simulated tidal volume pressure curves V_{t;sim(t)}: i.e., the model parameters are adapted in such a way that V_{t;sim(t)}=V_{t;exp(t)}. For this purpose, the patient is ventilated with one or more special maneuvers, which can be translated into a set of ventilation parameters θ_{b}. In the embodiment described herein, the vector-valued ventilation parameter θ_{b}={pᵢₙₛₚ,PEEP,tᵢₙₛₚ,tₑₓₚ,f,FiO₂, \dot{p}ᵤₚ,\dot{p}_{down}}. Further measured variables which are not directly linked to the ventilation device can also be included as parameters, such as, for example, the esophageal pressure. After this adjustment of the model parameters, the model is calibrated for a specific lung. The lung model can be patient-specific but can also be a model of a standardized lung or a mixed form, for example by averaging the lung data of a patient database. In this case, the possible parameters of the ventilation parameter are not exhausted in the values listed here. Rather, any type of ventilation curve can be parameterized with an arbitrarily large parameter set. For example, the pressure-time curve p_{trachea}(t)=fₚ(θ_{b}) can be parameterized. This pressure-time curve is then applied to the trachea in the form of a pressure boundary condition. Furthermore, the ventilation can also be varied with respect to other parameters, such as, for example, the position of the patient.

FIG. 1 shows a parameterized pressure-time curve. This curve is now taken into account as a boundary condition on the lung model, in the present embodiment on the Neumann-boundary of the differential equation system to be solved. Prior to this, the model parameters, for example the tissue stiffness, must be systematically adapted in the form of material parameters. Using this calibrated model, a physician is able to simulate a particular ventilation setting of the ventilation machine before adjusting those on the patient. Thus, before ventilating a patient who may be burdened with a lung disease, the physician can determine, with the aid of the model, how high, for example, the tissue expansions ε(x; t) would actually be for this individual scenario, i.e. the selected ventilation parameter θ_{b} in the lung of the patient, specifically before any possible damage to the lung due to overstretching, a barotrauma and/or frequent opening and closing of airways and alveoli or further damage mechanisms can occur as a result of incorrectly selected ventilation parameters. According to the current state of the art, a physician has no intuitive means for determining a more suitable set of ventilation parameters from the plurality “i” of possible settings θ_{b, i}, i.e. for determining an optimum ventilation parameter θ_{b, opt}.

### Optimization

In order to determine such an optimum ventilation parameter θ_{b, opt} from the multiplicity of possible ventilation parameters θ_{b,i} quality criteria are defined by means of which, in the present embodiment, an algorithm can be enabled to decide whether a selected ventilation parameter is more suitable for ventilation than another ventilation parameter.

An embodiment for the systematic improvement of the ventilation settings is shown below.

FIG. 2 shows an embodiment of the method for iteratively improving ventilation parameters within the framework of a mathematical optimization problem. Starting from the initial ventilation curve, which is defined by means of θ_{b, init}={pᵢₙₛₚ,PEEP,tᵢₙₛₚ,tₑₓₚ,f,FiO₂,\dot{p}ᵤₚ,\dot{p}_{down}}, the ventilation parameter θ_{b} is successively adapted in iterations “i” in the steps S2-S5. First, the settings initially selected in step S1 are tested in step S2, i.e. a simulation is carried out on the lung model with these settings. In the next step S3, the results of the model are evaluated. In one embodiment, this can be the calculation of strain values which serve as a measure of the mechanical load on the lung. In addition, the saturation of the patient's blood with oxygen and carbon dioxide is calculated on the basis of further output values of the model. This is done, for example, by evaluating the simulated oxygen partial pressure μ(O₂) and the carbon dioxide partial pressure μ(CO₂) in the venous or arterial blood of the patient. Reaching a predetermined gas saturation in the blood derived from the oxygen or carbon dioxide partial pressures is a compulsory condition in order to ensure physiologically meaningful simulation results. In the following step S4, an evaluation of the calculated output values takes place, as well as a selection of at least one next ventilation parameter. That is to say, in particular, a plurality of next respiration parameters can also be determined in parallel.

In the evaluation and selection step S4, it is checked, inter alia, whether the compulsory conditions or constrains are satisfied. For example, the oxygen content in the but is compared with the reference value. Alternatively and/or additionally, strain values calculated for the lung tissue can also be compared with predetermined strain maxima (reference values). Proceeding from an improvement function, a next ventilation parameter is proposed which fulfills the constrains.

The goal of a ventilation which is as optimal as possible, that is to say gentle on the patient, is achieved when the evaluation and selection of the optimization variables in step S4, at all locations x in the lung model, has reached a minimum or a predetermined quality measure or a predetermined reference value and, at the same time, the simulated oxygen saturation in the blood of the patient corresponds to a required minimum measure. The adaptation or improvement of the parameters in this case follows mathematical rules which are defined within the scope of the invention and are described in more detail below. If an optimum ventilation parameter θ_{b, opt} is found, it is output in step S6.

The optimization problem is described below with reference to an example algorithm. In general, a target function F is first defined as a function of specific output values of the model. During the optimization method, the function values of the target function are iteratively improved in such a way that they approach the minimum or a predetermined reference value or fall below it, for example. The selection method for determining a next ventilation parameter is based on the concept of approximating the function values of the target function B via a Gaussian process, so that an estimate for selecting the next suitable ventilation parameter can be made with the aid of the expected value of the Gaussian process and an acquisition function.

In the exemplary embodiment described here, the maximum tissue strain

$${max}_{x , t} ε ( x , t )$$

is optimized so that, with the optimum ventilation parameter, a predetermined maximum value for the maximum strain of the lung tissue is not exceeded, or alternatively a minimum of

$${max}_{x , t} ε ( x , t )$$

with respect to Θ_{b}, is found. The function B to be optimized is thus not explicitly known, since there is no analytical expression of the output value. For example, values for the tissue strain ε(x, t) are only accessible via the simulation.

The optimization takes place taking into account constraints, for example the oxygen saturation in the blood of the patient, for which there is likewise no simple analytical expression or relationship to the input variables of the model and whose fulfillment can therefore likewise only be evaluated with the aid of the model.

In one embodiment of the patient-specific lung model for determining optimum ventilation settings, there are therefore two functions which are used for evaluating the result values of the lung model. On the one hand, a function B, which describes the mechanical loading of the lung by ventilation B(ε(\vec{x}, t),p(\vec{x}, t)). The mechanical load is understood here as a function of pressure and tissue strain, but in another embodiment, it can also include the collapse or re-opening of parts of the lung. On the other hand, a function N, which serves to evaluate the fulfilment of a constrain N(μ_{CO}_{₂}(\vec{x}, t), μ_{O}_{₂}(\vec{x}, t)). Both functions are dependent on the input variables of the model {pᵢₙₛₚ,pᵢₙₛₚ,PEEP,tᵢₙₛₚ,tₑₓₚ,f,FiO₂,\dot{p}ᵤₚ,\dot{p}_{down}, . . . }.

The problem can in principle be formulated as a nonlinear optimization problem “O” without constraints, i.e. min(O(B, N)), or as a nonlinear problem with constraints, i.e. min(B), N>b. Where b represents a lower limit for, for example, the oxygen saturation. In the embodiment described below and particularly preferred, the optimization problem is formulated as a nonlinear optimization problem with a constraint, which is solved according to the Bayesian optimization approach, as it is described in “Gardner et al., Bayesian Optimization with Inequality Constraints” Proceedings of the 31st International Conference on Machine Learning, Beijing China. JMLR: W&CP volume 32”.

The advantages of Bayesian Optimization in the context of optimizing the ventilation parameter of a lung machine are its properties as a global optimization method. A further advantage for the present application is seen in the fact that no gradient of the function to be optimized is required, or the method does not have to approximate this by means of a finite difference method. The greatest advantage of Bayesian optimization for the present optimization problem is seen in the fact that the method is very efficient, i.e. relatively few model evaluations are required in order to provide an optimized parameter, and the method can be further parallelized for further increase in efficiency. Consequently, optimized ventilation parameters can be supplied to a patient in a short time, for example after a request from a clinic to a service provider providing this optimization model.

In the embodiment described herein, an “optimal” parameter θ_{b, opt} is defined as follows. The amount of oxygen transported from the lungs into the patient's body must be such that the oxygen saturation in the blood is above a predetermined reference value. This is defined, for example, as a threshold value with, for example, S_{O2}=90%, but can also be lower or higher in other scenarios. This constrain for solving the optimization problem is summarized in N(μ_{CO}_{₂}(\vec{x},t),μ_{O}_{₂}(\vec{x}, t)). In FIG. 2 this condition is checked in step S3 “Evaluation of the lung model”. Furthermore, in the case of the “optimum” parameter θ_{b, opt} for example, the maximum strain of the ACs εₘₐₓ in the lung of the patient should be as small as possible. In this case, for example, the absolute maximum of the strain occurring in the lung is evaluated, for example both with respect to the location x and with respect to the time t. For the evaluation, at least one complete breath is considered. Since the elasticity model of the ACs used in the lung model is a dimension-reduced model with the dimension zero (“D-0”), the strain is not a tensor but a scalar. This reduces the computing time, or the computing power required for evaluation. The so-called volumetric strain of the ACs is considered, i.e. the ratio between the initial volume and the inflated volume is calculated. In the embodiment variant described here, the function B to be optimized would accordingly be defined as

$$B = {max}_{x ∈ Ω , t ∈ T} ε ( \vec{x} , t )$$

The introduced mathematical functions B and N thus define an optimization problem with respect to the ventilation parameter θ_{b} and not with respect to the model parameter θₘ. The values of the function B are now optimized to a predetermined condition, taking into account the constrain N≥SO₂, i.e. the values of the function N(θ_{b}).

The algorithm for finding an optimal ventilation parameter θ_{b, opt} is based essentially on two parts: a method for creating probabilistic regression models and a so-called acquisition function. The concept of Bayesian optimization is used to solve the following optimization problem:

$${θ}_{b , opt} = arg {min}_{N ( {θ}_{b} ) > λ )} B ( {θ}_{b} )$$

FIG. 3 schematically shows the sequence of an optimization loop. In a first step a set of initial data points {θ_{b,init,1:J}} is determined by means of a Monte Carlo or Latin hypercube sampling. The set comprises, for example, J=30 different ventilation parameters θ_{b,init}. The simulation model is run through with these statistically determined ventilation parameters. The output variables of the model are then used in step i) for the calculation of the defined functions B and N. This results in a statistical distribution of the respective 30 function values of N and B. Based on the set of initial data points {θ_{b,init,1:J},B(θ_{b,init,1:J}),N(θ_{b,init,1:J}}, two Gaussian processes are trained. Subsequently, further promising points in the parameter space are successively selected with an acquisition function, the simulation model is evaluated, and the Gaussian process models are subsequently improved with the aid of the new data points. This process is repeated for a certain number [1, K]:={k∈ [symbol] |1≤k≤K} of iterations. Consequently, Bayesian optimization always iterates between training regression models and using them to predict promising candidates for the optimum. In this case, in the embodiment described here, based on the initial set of J=30 ventilation parameters, in each case only one further ventilation parameter θ_{b,next} is determined and used for the next simulation. In the embodiment shown here, the regression models then comprise this further ventilation parameter, i.e. the models become more and more accurate from iteration to iteration, since the evaluation is based on more and more data points, as a result of which the informative value is improved.

In the following, the regression approach used is described first and then the acquisition function used.

First, the assumption is made that the function B to be optimized and the constrain N can each be modeled as a Gaussian process, i.e. N,B˜GP(μ(⋅), k(⋅)). Here μ(0_{b}) = [symbol] [B(⋅)] represents the mean value function or the expected value [symbol] und k(⋅) the covariance function of the Gaussian process, which is defined as follows:

k(θ_{b},θ_{b}′)= [symbol] [(B(θ_{b})−μ(θ_{b}))(B(θ_{b}′)−μ(θ_{b}′))]

As a consequence of the modeling as a Gaussian process, given a set of given input variables Φ={θ_{b,1}, θ_{b,2}, . . . , θ_{b,H}} and associated function values of B(Φ)={Bθ_{b,1}), B(θ_{b,2}), . . . , B(θ_{b,H})}, the following a posterior probability distribution results for a new test point θ_{b,next}: \tilde{B}(θ_{b,next})˜p(B(θ_{b,next})|θ_{b,next}, Φ, B(Φ)).

Since this is a Gaussian distribution, it can be characterized by the mean value and the variance:

μ_{B}(θ_{n,next})=μ(θ_{b,next})+k(θ_{b,next}, Φ)k(Φ, Φ)⁻¹(B(Φ)−μ(Φ))

Σ_{B}²(θ_{b,next})=k(θ_{b,next},θ_{b,next})−k(θ_{b,next},Φ)k(Φ, Φ)⁻¹k(Φ, θ_{b,next}).

By means of the formulas shown, it is therefore now possible to update a posterior distribution for any point with the aid of new data. The described regression method is now used to train two regression models: one for N(⋅) and one for B(⋅).

### Acquisition Function

The most important step in Bayesian optimization is the determination of the next candidate or candidates θ_{b,next}. This is done with the aid of the acquisition function. Here, the so-called “Expected—Constrained—Improvement Function” is used. This modified acquisition function can be derived from the “Expected—Improvement—Function” as follows. This process is described in “Gardner et al., Bayesian Optimization with Inequality Constraints” Proceedings of the 31st International Conference on Machine Learning, Beijing China. JMLR: W&CP Volume 32,” in detail. First, the so far best point in the data set T_{B} evaluated so far is defined as θ_{b}⁺. Now, the improvement achieved by a new candidate can be expressed as:

Ĩ(θ_{b})=max{0, B(θ_{b+})−\tilde{B}(θ_{b,next})}

The expected value of the improvement then results as follows: EI(θ_{b,next})= [symbol] [Ĩ(θ_{b,next})|θ_{b,next}]. After conversion, the following analytical expression EI(θ_{b,next})=Σ_{N}(θ_{b,next})(ZΦ(Z)+φ(Z)), is obtained, wherein Φ is representing the cumulative distribution function and φ is representing the density function of a standard normal distribution. In addition,

$$Z = \frac{{μ}_{B} ( {θ}_{b , next} ) - B ( {θ}_{b}^{+} )}{{\sum }_{N} ( {θ}_{b , next} )} .$$

In order to satisfy the constraints, namely that, according to the embodiment described here, the oxygen saturation S_{O2} must not fall below a lower threshold, an extension of the proposed acquisition function according to Gardner et al. is proposed. The so-called “constraint improvement” for a candidate θ_{b,next} is therefore:

I_{C}(θ_{b,next})=Δ(θ_{b,next})max{0,B(θ_{b}⁺)−B(θ_{b,next})}=Δ(θ_{b,next})I(θ_{b,next}),

wherein Δ(θ_{b,next})∈{0,1} represents an indicator function, which is 1 if N(θ_{b,next})≥λ and zero otherwise, i.e. the indicator function sorts out all possible ventilation parameter by multiplication by zero if the constrain λ of the oxygen saturation is not satisfied. Since the calculation of N(θ_{b,next}) is just as computationally complex as the calculation of B(θ_{b,next}), a surrogate model in the form of a Gaussian process is also introduced for N(θ_{b,next}) and is used instead of the simulation model. That is to say, the Gaussian model is a surrogate model, and the search for a new candidate is out-sourced to the surrogate model. This is done by searching for the minimum/maximum of the acquisition function. Due to the Gaussian marginal distribution of \tilde{B}(θ_{b,next}) the following expression is obtained for the “Expected—Constrained Improvement” acquisition function, which is used in the optimization:

EI_{C}(θ_{b,next})=PF(θ_{b,next})EI(θ_{b,next})

where PF(θ_{b,next}) is defined as:

PF(θ_{b,next}):=Pr[Ñ(θ_{b,next})≤λ].

FIG. 3 schematically shows the sequence of an optimization loop. The actual optimization loop relates to method steps i) to iii). First, a ventilation parameter is defined which is to be optimized. Here, for example, the following five parameters are selected for defining a vector-valued ventilation parameter θ_{b} ge\ddot{w}ahlt: θ_{b}={PEEP,pᵢₙₛₚ,tᵢₙₛₚ,tₑₓₚ,FiO₂}.

An initial Latin hypercube design with 30 candidates {θ_{b init,1:30}} is now created for this ventilation parameter. The simulation model is then evaluated for these 30 points, i.e. 30 simulation runs are made and the value of the optimization function B and of the constraint N is calculated. Correspondingly, the initial data set T={θ_{b,1:30}, N(θ_{b,1:30})/B(θ_{b,1:30})} now exists. On the basis of this data set T, a Gaussian process model is now trained in each case. Then the following three steps i) to iii) schematically illustrated in FIG. 3 are repeated, for example K=20 times, in the sequence indicated, wherein step ii) is to be started here:

Step ii): With the aid of the acquisition function, a new candidate θ_{b,next} is calculated or selected in step ii) “Evaluation and selection”. Likewise, the calculated function values B(θ_{b,next}) can be evaluated in order to check whether a predetermined reference variable of the mechanical load B(ε(θ_{b,next});p(θ_{b,next})) of the lung is satisfied by the current ventilation parameter, or whether a predetermined maximum number of iterations has been reached.

Step iii): The next candidate θ_{b,next} proposed in step ii) is transferred to the model for recalculation or simulation.

Step i): The calculation of N(θ_{b,next}), B(θ_{b,next}) follows.

At the end of the algorithm, i.e. of the optimization method, that parameter θ_{b,opt} is returned for which the constraint is satisfied and for which the optimization function has the lowest value, i.e. comprises the lowest mechanical load.

In this embodiment, the algorithm ends the optimization after K=20 runs. K is determined by the user for this purpose. In this case, the user can additionally also use an abort criterion, the method being terminated automatically, and the ventilation parameter being output to the ventilation machine by finding a first ventilation parameter which satisfies the conditions.

FIG. 4 schematically shows a preferred embodiment of the system for the patient-specific determination of an optimum ventilation parameter. The individual steps of FIG. 4a are described as follows: 4a_1: imaging, acquisition of additional data; 4a_2: release, pseudonymization; 4a_3: data transfer from clinic server to external, for example to a cloud computing environment; 4a_4: calculation model generation; 4a_5: optimization of the ventilation settings or finding of θ_{b,opt} respectively; 4a_6: data transfer PEEP, Pᵢₙₛₚ, f, tᵢₙₛₚ, tₑₓₚ, FiO₂, etc.; 4a_7: authorization; 4a_8: application. In the embodiment shown here in FIG. 4 the model is constructed in a patient-specific manner by means of data from a computer tomograph. In addition, a patient-specific ventilation curve is supplied to the model for patient-specific calibration. The patient-specific data are transmitted digitally from the clinic to an external computing environment, which is provided, for example, in the form of a computing server at a cloud computing provider or as a local computing server. Alternatively, only the model calculation, i.e. the computation-intensive part of the model, could be performed by a cloud computing provider and the actual optimization of the ventilation parameters could be performed, for example, by a further provider or in the clinic. In particular, this is to be understood as meaning so-called “software as a service” concepts which provide a clinic or a physician with the optimized ventilation parameters of the patient for operating the ventilation device. These can then be based on patient-specific data, that is to say for example on data obtained by means of CT data, respiration measurements or result from a patient-specific database, or from a general patient database which typifies the preexisting disease.

The specifically optimal ventilation parameter determined by the model for the patient is then digitally returned to the clinic. From there, the data reach a ventilator, which is configured by means of the calculated optimal ventilation parameter.

Alternatively, the model can also be implemented directly on a computing unit of the ventilator, as schematically illustrated in FIG. 4 The individual steps of FIG. 4b are described as follows: 4b_1: imaging, acquisition of additional data; 4b_2: release, pseudonymization; 4b_3: data access to clinic server of respirator or in clinic; 4b_4: computer model generation; 4b_5: optimization of respiratory settings or finding θ_{b,opt} respectively; 4b_6: data provision PEEP, pᵢₙₛₚ, f, tᵢₙₛₚ, tₑₓₚ, FiO₂, etc.; 4b_7: authorization; 4b_8: application. Here, the patient-specific data, at least comprising the evaluated computer tomographic images of the patient's lung, are transmitted from a hospital server directly to the ventilation machine. The ventilation machine can then fully automatically ensure optimum patient-specific ventilation by performing the calculation of the optimum parameters on a computer unit of the ventilation machine. This means that the lung model can be implemented on a computing unit of the ventilator, so that simulation is carried out on the ventilation machine, or alternatively the ventilator ensures that an external computing unit is used for the simulation and the selection of the next ventilation parameter(s) is carried out on the ventilation machine, i.e. in particular the simulation step ii) for evaluating and selecting the next ventilation parameter(s).

In a further alternative shown in FIG. 4c, the model is implemented on a simulation server which is located in the clinic, as is the computer tomograph and the ventilation machine. The individual steps of FIG. 4c are described as follows: 4c_1: imaging, acquisition of additional data; 4c_2: release, pseudonymization; 4c_3: data access to hospital servers; 4c_4: computer model generation; 4c_5: optimization of the ventilation settings or finding θ_{b,opt} respectively; 4c_6: data transfer, data access PEEP, pᵢₙₛₚ, f, tᵢₙₛₚ, tₑₓₚ, FiO₂, etc.; 4c_7: authorization; 4c_8: application. The patient-specific data, e.g. image data, are supplied to this simulation server. The simulation server can process the image data into 3D structural data on which the lung model is based, for example by means of an algorithm based on artificial intelligence or machine learning. The optimization and simulation steps by which the optimal parameter is determined are performed on the simulation server of the clinic and are delivered afterwards to the ventilation device.

FIG. 5 shows an embodiment of a system 100 having the following components: a data processing unit 120, a ventilation machine 130, a computer tomograph (CT) 140 and a server 150. The listed units are networked with one another, so that digital data can be exchanged between the units of the system 100, in the sense of a data logistic process chain. The components of the system 100 can operate locally separated from one another at different locations, as is indicated by the dashed lines 160. That is to say, the components of the system form a network which makes it possible to exchange digital data with one another. In particular, taking into account an encrypted, for example personalized, access, in particular patient-specific data, when exchanging the data between the components of the system 100. For example, the CT 140 is located in a clinic A and the server 150 is located in a further clinic B or at a service provider, for example a provider of a computing cloud. The ventilation machine 130 is located within the same clinic A as the CT 140 or at the same clinic B as the server 150 or at another clinic C. In a preferred embodiment, the CT 140 is securely located in the clinic A together with the ventilation machine 130. The server 150 is also located within the clinic A. The server 150 comprises at least one memory unit 152 and at least one computing unit (CPU) 151. The server 150 transfers the patient-specific “CT” image data on request, for example by requesting the data processing device 120 to the data processing device 120. The data processing device 120 has an operating unit 121 and a computing unit 124. The operating unit 121 comprises a processor (CPU) as well as a memory unit. The operating unit 121 is implemented, for example, by a personal computer and allows a user to initialize the request for the transfer of patient-specific data. Furthermore, the operating unit 121 enables the user to start the optimization in order to find optimum ventilation settings. The computing unit 122 of the data processing device 120 can be locally separate from the operating unit. The computing unit 122 is ideally embodied by a server which, in particular, has a high computing capacity. The computing unit therefore comprises at least one CPU 123 and at least one memory unit 124, wherein in this preferred embodiment, the lung model is implemented on the computing unit 122 or can be installed on the latter from outside the data processing device 120.

The operating unit can also be part of the server 122. A user receives a set of CT image data of the server 150 via the operating unit 121. From the operating unit 122, the set of image data is forwarded to the computing unit 122, and the user initializes, via the operating unit 121, the evaluation of the set of CT image data in order to provide to the model the 3D geometric structure data set of the lung necessary for modeling. In addition, the user receives or downloads a patient-specific ventilation curve from the server 150 via the operating unit 121. In particular, this process can also be carried out automatically by the software. Alternatively, the ventilation curve can also be downloaded from any desired patient database. After calibrating the model with the ventilation curve, the user starts the simulation, which ideally automatically provides the optimum ventilation parameter and delivers them to the ventilation machine 130 via the server 150.

In an alternative embodiment, the computing unit 122 can be outsourced, for example, to a service provider which provides a high calculation capacity, in particular for simulating the lung model, wherein, in particular, a plurality of optimum ventilation parame-ters can be calculated in parallel, for example by multiple, parallel selection of next candidates by means of one or more acquisition functions. In a further preferred embodiment, the computing unit 150, which is embodied, for example, as a simulation server, likewise comprises an algorithm for providing the structural data from the transferred, patient-specific CT data, in particular based on an artificial intelligence for recognizing relevant lung-specific features from the CT slice images of a patient. The server 150 transfers this data to the computing unit 122. Alternatively, the simulation server 150 can also carry out the simulation of the lung model if the latter is equipped with corresponding computing capacity, or can access it, and the optimization of the ventilation parameters is carried out by an external service provider which comprises the data processing unit 120.

In FIG. 6 a preferred embodiment of a ventilation machine 230 is schematically illustrated. The ventilation machine 230 comprises a control unit 235 for controlling the ventilation by means of ventilation parameters. Furthermore, the ventilation machine 230 comprises a data processing device 231. As indicated by the dashed lines 260 the ventilation machine 230 is spatially separated from a server 250 and the CT 240. For example, the CT 240 and the server 250 are located together in a clinic A, and the ventilation machine 230 may be located in a further clinic B. Together with the CT and the server, the ventilation machine forms a network for exchanging data, in particular patient-specific data, comprising the access rights for transmitting the patient-specific information which are connected to the data exchange.

The ventilation machine 230 comprises a data processing device 231, which in turn comprises an operating unit 234, with which a user, for example a physician, can operate the ventilation machine and, in particular, can initialize the determination of optium ventilation parameters and, in particular, can generate and evaluate the at least one ventilation curve for calibrating the lung model. In this particularly preferred embodiment, the lung model and the optimization method are implemented on the ventilation machine, so that the optimization steps for finding optimum patient-specific data can be carried out or coordinated at least partially or completely on the ventilation machine. For example, the ventilation machine 230 may also outsource data records or calculations to an external data server configured for performing lung simulations. For this purpose, the ventilation machine comprises a computing unit 232 with a memory unit 235 and at least one processor (CPU) which has a high computing power, so that the optimization steps i)-iii), comprising the provision of the initial parameters by means of Monte Carlo or Latin hypercube sampling, can be carried out.

In addition, the data processing device can ideally prepare the 3D CT image data for the required structural geometry of the lung model. In a specific application, the ventilation machine 230 is located in a hospital in which both the CT 240 and the server 250 are located. At the request of a physician via the operating unit 234 of the ventilating machine, the patient-specific CT image data are transferred to the machine 230 from the tomograph 240 via the server 250 or are already located on the server 250 and are then structurally prepared for the model directly by the data processing device 231 of the ventilating machine. Ideally, a ventilation curve of the patient for calibrating the model on the patient can be recorded and evaluated in parallel with the ventilation machine 230. The software implemented on the ventilation machine 230 then processes the 3D CT image data provided in the meantime and digitally builds up the 3D geometry of the lung image. Ideally, a calibration by means of the ventilation curve is carried out automatically by the computing unit 232. A physician can then carry out the optimization on the ventilating machine 230, so that the optimum ventilating parameter is then delivered directly to the control unit 235 of the ventilating machine 230.

FIG. 7 shows a further embodiment of the invention in which no imaging takes place. Instead, the operator makes a ventilation proposal which comprises at least two ventilation parameters and which can at the same time be regarded as a signal for release and pseudonym formation for the exchange of patient-specific data. The individual steps of FIG. 7 are described as follows: 7_1: ventilation proposal PEEP, pᵢₙₛₚ, f, tᵢₙₛₚ, tₑₓₚ, FiO₂, etc.; 7_2: data transfer PEEP, pᵢₙₛₚ, f tᵢₙₛₚ, tₑₓₚ, FiO₂, etc.; 7_3: calculation model generation; 7_4: model evaluation; 7_5: data transfer, feedback regarding the effects of the settings on the patient's lung, e.g. max. strain, O2- perfusion, etc.; 7_6: decision yes no; 7_7: application if necessary. The data of the ventilation parameter proposal, for example of a physician, which in this case can also be located at a different location from that of the ventilation machine, are then transmitted to the storage or calculation location, usually to a cloud server for the simulation and optimization. In this case, the ventilation device can ideally be configured by the external operator via the network by means of the ventilation parameter proposal and can be monitored with regard to the result achieved in the form of, for example, patient-sensitive lung data, such as tissue strain. In this embodiment, the external operator can abort the optimization, in particular after a number of iterations. In addition to the proposed ventilation parameters, additional patient data of other embodiments can ideally also be transmitted in order to generate or personalize the calculation model of the lung, for example structure-geometric data of the lung of the patient. In the subsequent step, at least one model evaluation takes place, which generates result data in the form of fluid and structural mechanical values, data for gas exchange, chemical reactions and other infor-mation that can be determined with the calculation model of the lung. In this case, a first evaluation results in a point-by-point interrogation of the lung behavior with respect to the ventilation proposal, whereupon the optimization generates further ideal ventilation parameters and indicates them to the external operator with respect to their patient-specific effect, for example as a continuously, with each iteration expanding graphic, of one or more patient-relevant values. In this way, the model can present the user with a counterproposal based on his proposal.

### REFERENCE NUMERALS AND VARIABLES

100, 200 System

120, 231 Data processing device

121, 234 Operating unit

122, 232 Computing unit

123, 233 Processor (CPU)

124, 235 Memory unit

130, 230 Ventilation machine

140, 240 Computer tomograph (CT)

160, 260 Spatial separation line

150, 250 Server

151, 251 Computing unit

152, 252 Memory unit

| Index/Subscript i | Size in iteration step i |
|---|---|
| J | Size/number of initial ventilation |
| | parameters |
| K | Number of iteration steps |
| θ_{b} | Ventilation parameters (vector with |
| | p_insp, PEEP etc) |
| θ_{b, next} | Proposed by the selection procedure, |
| | next ventilation parameter. |
| θ_{b, i} | Ventilation parameter in iteration step i |
| θ_{b, opt} | Optimum ventilation parameters |
| θ_{b, init} | Inital ventilation parameter(s) |
| {θ_{b, init, 1:30}} | Set of 30 initial ventilation parameters |
| {θ_{b, init, 1:J}} | Set of J initial ventilation parameters |
| θ_{b}⁺ | Best ventilation parameter so far (in |
| | Bayesian optimization) |

1. A method for automatically determining at least one optimal ventilation parameter θ_{b, opt} for operating a ventilation machine, comprising the computer-implemented steps of:
Providing a patient-specific digital lung model, inputting at least one initial input ventilation parameter θ_{b, init} as ventilation parameter θ_{b, i}=θ_{b, init};
Performing the following steps (i) to (iii) iteratively until a completion criterion is met which checks the reaching of an optimal patient-specific ventilation parameter θ_{b, i}=θ_{b, opt} and/or checks the reaching of a predetermined number of iterations;
i) Evaluation of the mechanical ventilation simulated on the lung model as a function of the ventilation parameter θ_{b, i} by determining the value of at least one patient-specific target function F=F(θ_{b,i}) from the lung model,
wherein
the target function F describes a lung reaction to the simulated mechanical ventilation as a function of at least one output parameter of the lung model,
ii) Evaluating the at least one determined value of the function F on the basis of at least one predetermined reference value, and
selecting at least one next ventilation parameter θ_{b, next}, using a selection method dependent on at least one previously used ventilation parameter θ_{b,i};
iii) Using the at least one next ventilation parameter θ_{b, next} as ventilation parameter θ_{b,i} to determine F=F(θ_{b,i}=θ_{b,next}) in step i);
Providing the patient-specific optimal ventilation parameter θ_{b, opt.} if the completion criterion is met.

2. (canceled)

3. The method according to claim 1, wherein the selection method for selecting at least one next ventilation parameter θ_{b, next} includes an algorithm, in particular an optimization method according to Bayes, which is implemented in particular using one or more Gaussian processes, random Forrests, artificial neural networks or other regression models, a fuzzy logic algorithm, an algorithm based on an evolutionary method, an algorithm including a gradient method, and/or an algorithm based on stochastic techniques.

4. The method according to claim 1, wherein the selection method includes an acquisition function for selecting at least one next ventilation parameter θ_{b, next} using a probabilistic regression method which depends on at least one previously determined data set Ti=(θ_{b,i}, F(θ_{b,i})), in particular a regression method for a Gaussian process.

5. The method according to claim 1, wherein the selection method for selecting at least one next ventilation parameter θ_{b, next} includes an acquisition function which uses the expected value of the improvement, in particular taking into account constrains.

6. The method according to claim 1, wherein the selection method for selecting at least one next ventilation parameter θ_{b, next} includes an acquisition function which uses an entropy search, or which uses a knowledge gradient.

7. (canceled)

8. The method according to claim 1 wherein it is provided in step (ii) to determine at least one next ventilation parameter θ_{b,next}, wherein the selection process takes place on a computing unit, in particular a processor, and the model evaluations are carried out in parallel on other computing units and the results are subsequently recombined.

9. (canceled)

10. (canceled)

11. (canceled)

12. The method according to claim 1, wherein step (ii) of the method includes that, as a patient-specific reference value, a maximum strain Bₘₐₓ(ε(x, t)) and/or a maximum pressure Bₘₐₓ(p(x, t)) of the lung must not be exceeded, and/or an oxygen saturation S_{O2} must not be undershot.

13. The method according to claim 1, wherein in step (iii) the ventilation parameter values θ_{b, i} are varied stochastically.

14. The method according to claim 1, wherein a set of initial input respiration parameter values θ_{b,init,1:J} is produced by means of a random or quasi-random method, in particular Monte-Carlo or Latin hyper-cube sampling.

15. The method according to claim 14, characterized in that, in step (ii), the function values of function B which have been calculated from the at least one output parameter by simulating the set of initial input ventilation parameters θ_{b, init,1:J} are used for training a Gaussian process, wherein B describes a mechanical loading of the lung as a function of at least one output parameter of the lung model, wherein the at least one output parameter describes a mechanical loading value of the lung of the patient.

16. The method according to claim 1, characterized in that, in step (ii), the function values of function B which have been calculated from the at least one output parameter by simulating the set of next input ventilation parameters θ_{b, next,1:n} are used for further training of the Gaussian process, wherein B describes a mechanical loading of the lung as a function of at least one output parameter of the lung model, wherein the at least one output parameter describes a mechanical loading value of the lung of the patient.

17. The method according to claim 15, characterized in that, in step (ii), the selection function is an acquisition function, which calculates next ventilation parameter values θ_{b, next} taking into account the following: I(θ_{b,next})=Δ(θ_{b,next})*max{0, B(θ_{b}⁺)−B(θ_{b,next})}, where B(θ_{b}⁺) represents the function value with the so far lowest mechanical load as a function of the ventilation parameter θ_{b}⁺ which has been most suitable so far, and wherein the indicator function is 1 if the function N(θ_{b, next}) is less than or greater than a predetermined reference value, and zero otherwise, wherein N describes the enrichment of gas in the blood of the lung as a function of at least one output parameter of the lung model, wherein the at least one output parameter describes a gas partial pressure in the blood of the patient, and/or B describes a mechanical loading of the lung as a function of at least one output parameter of the lung model, wherein the at least one output parameter describes a mechanical loading value of the lung of the patient.

18. The method according to claim 1, characterized in that the patient-specific lung model is produced as a function of measured image data of the lung of the patient.

19. The method according to claim 1, characterized in that, before the method is started, the patient-specific lung model is calibrated by means of a ventilation curve which contains either a pressure-time curve p_{trachea}(t) and/or a flow-time curve Q_{trachea}(t) and/or a volume-time curve v_{trachea}(t) and/or respiratory gas mixture composition-time curve or curves of the patient derived therefrom, which comprises at least one breath of the patient.

20. The method according to claim 19, characterized in that the parameterized pressure-time curve p_{trachea}(t) maps the patient-specific pressure in the trachea of the patient-specific lung model.

21. (canceled)

22. A computer program product using a digital lung model, comprising instructions which, when executed on a processor of a data processing unit, cause the following steps (i) through (iii) to be performed iteratively until a completion criterion is met which tests for the achievement of optimal patient-specific ventilation parameters θ_{b,i}=θ_{b,opt} and/or provides for the achievement of a predetermined number of iterations:
i) Evaluation of the mechanical ventilation simulated on the lung model as a function of the ventilation parameter θ_{b, i}, by determining the value of at least one patient-specific target function F=F(θ_{b,i}) from the lung model,
wherein
the target function F describes a lung reaction to the simulated mechanical ventilation as a function of at least one output parameter of the lung model, ii) Evaluating the at least one determined value of the function F on the basis of at least one predetermined reference value, and selecting at least one next ventilation parameter θ_{b,next}, using a selection method dependent on at least one previously used ventilation parameter θ_{b,i};
iii) Using the at least one next ventilation parameter θ_{b,next} as ventilation parameter θ_{b, i} to determine F=F(θ_{b,i}=θ_{b,next}) in step i).

23. A computer readable medium having stored thereon a computer program product utilizing a digital lung model and comprising instructions which, when executed on a processor of a data processing unit, cause the following steps (i) through (iii) to be performed iteratively, until a completion criterion is met that tests for achievement of optimal patient-specific ventilation parameters θ_{b,i}=θ_{b, opt} and/or provides for achievement of a predetermined number of iterations:
i) Evaluation of the mechanical ventilation simulated on the lung model as a function of the ventilation parameter θ_{b, i}, by determining the value of at least one patient-specific target function F=F(θ_{b,i}) from the lung model,
wherein
the target function F describes a lung reaction to the simulated mechanical ventilation as a function of at least one output parameter of the lung model,
ii) Evaluating the at least one determined value of the function F on the basis of at least one predetermined reference value, and
selecting at least one next ventilation parameter θ_{b,next}, using a selection method dependent on at least one previously used ventilation parameter θ_{b,i};
iii) Using the at least one next ventilation parameter θ_{b,next} as ventilation parameter θ_{b, i} to determine F=F(θ_{b,i}=θ_{b,next}) in step i).

24. A system comprising at least one data processing device and a computer program product, wherein
the at least one data processing device is configured to execute the computer program product and, in particular, to exchange data with a ventilation machine for controlling the ventilation machine, wherein the computer program product uses a digital lung model, and wherein the computer program product comprises instructions which, when executed on a processor of the data processing device, cause the following steps (i) to (iii) to be performed iteratively until a completion criterion is met, which checks for achievement of optimal patient-specific ventilation parameters θ_{b,i}=θ_{b, opt} and/or provides for achievement of a predetermined number of iterations:
i) Evaluation of the mechanical ventilation simulated on the lung model as a function of the ventilation parameter θ_{b, i}, by determining the value of at least one patient-specific target function F=F(θ_{b,i}) from the lung model,
wherein
the target function F describes a lung reaction to the simulated mechanical ventilation as a function of at least one output parameter of the lung model,
ii) Evaluating the at least one determined value of the function F on the basis of at least one predetermined reference value, and
Selecting at least one next ventilation parameter θ_{b,next}, using a selection method dependent on at least one previously used ventilation parameter θ_{b,i};
iii) Using the at least one next ventilation parameter θ_{b,next} as ventilation parameter θ_{b, i} to determine F=F(θ_{b,i}=θ_{b,next}) in step i).

25. Ventilation machine comprising at least a control unit and a data processing device adapted to read and execute at least one computer program product, and wherein the at least one data processing device is configured to provide data to the control unit for controlling the ventilation machine and/or to exchange data with the control unit for controlling ventilation of a patient, wherein the computer program product uses a digital lung model, and the computer program product comprises instructions which, when executed on a processor of the data processing device, cause the following steps (i) to (iii) to be performed iteratively until a completion criterion is met which tests for achievement of optimal patient-specific ventilation parameters θ_{b,i}=θ_{b, opt} and/or provides for achievement of a predetermined number of iterations:
i) Evaluation of the mechanical ventilation simulated on the lung model as a function of the ventilation parameter θ_{b, i}, by determining the value of at least one patient-specific target function F=Fθ_{b,i}) from the lung model,
wherein
the target function F describes a lung reaction to the simulated mechanical ventilation as a function of at least one output parameter of the lung model,
ii) Evaluating the at least one determined value of the function F on the basis of at least one predetermined reference value, and
Selecting at least one next ventilation parameter θ_{b,next}, using a selection method dependent on at least one previously used ventilation parameter θ_{b,i};
iii) Using the at least one next ventilation parameter θ_{b,next} as ventilation parameter θ_{b, i} to determine F=F(θ_{b,i}=θ_{b,next}) in step i).
